(19) United States
(12) Patent Application Publication
(10) Pub. No.: US 2025/0189607 A1
(43) Pub. Date: Jun. 12, 2025

(54) MAGNETIC RESONANCE SYSTEM AND SCANNING METHOD BASED ON MAGNETIC RESONANCE SYSTEM

(71) Applicant: GE Precision Healthcare LLC, Waukesha, WI (US)

(72) Inventors: Yuechen Liu, Beijing (CN); Kun Wang, Beijing (CN); Yaan Ge, Beijing (CN); Fan Yang, Beijing (CN); Yanting Huo, Beijing (CN)

(21) Appl. No.: 18/958,808

(22) Filed: Nov. 25, 2024

(30) Foreign Application Priority Data
Dec. 8, 2023 (CN) 202311681377.3

(51) Int. Cl. G01R 33/28; G01R 33/54

(52) U.S. Cl. CPC G01R 33/288; G01R 33/543

(57) ABSTRACT

A magnetic resonance system and a scanning method based on a magnetic resonance system are presented. The method includes: for at least one scanning parameter, acquiring a numerical distribution map determined on the basis of the magnetic resonance system; determining an implant-related sensitive region of a subject to be scanned; determining, from the numerical distribution map, a first numerical value corresponding to the sensitive region; acquiring a limit value of the at least one scanning parameter limited by an implant of the subject; adjusting the limit value of the at least one scanning parameter on the basis of the first numerical value; and scanning the subject by using the adjusted limit value of the at least one scanning parameter.

### CROSS REFERENCE TO RELATED APPLICATIONS

The present application claims priority and benefit of Chinese Patent Application No. 202311681377.3 filed on Dec. 8, 2023, which is incorporated herein by reference in its entirety.

### TECHNICAL FIELD

Embodiments of the present invention generally relate to medical imaging technology, and relate in particular to a magnetic resonance (MR) system and a scanning method based on a magnetic resonance system.

### BACKGROUND

Magnetic resonance imaging technology has been widely used in the field of medical diagnostics. A magnetic resonance system usually has a main magnet, a radio frequency system, a gradient system, and a computer system. The main magnet is used to generate a main magnetic field, and a subject to be scanned is positioned in the space of the main magnetic field to receive a magnetic resonance scan. The radio frequency system is used to generate radio frequency pulses to excite the subject to generate magnetic resonance signals. The gradient system is used to generate gradient fields superimposed on the main magnetic field such that the magnetic resonance signals have encoded information. The magnetic resonance signals having the encoded information are converted into digital image signals, which undergo signal processing by a computer to reconstruct a medical image of the subject.

Typically, on the basis of different clinical diagnostic requirements, corresponding scanning parameters are selected to perform a magnetic resonance scan, and these parameters may involve a magnetic field, a gradient, a radio frequency, a reconstruction method, etc. For a subject to be scanned having an implant, it is also necessary to refer to a corresponding implant safety manual during configuration of scanning parameters, wherein safety limit values for scanning parameters to which an implant product can be adapted during a magnetic resonance scan are specified. The limit values are usually determined by implant manufacturers according to the characteristics of their own implant products in combination with magnetic resonance simulation and emulation environments, a separate (or single) gradient field or radio-frequency field environment, or comprehensive product parameters of each magnetic resonance product manufacturer.

Furthermore, industry standards are also evolving for magnetic resonance scanning of patients having implants. For example, safety limit values for various scanning parameters for implants are specified in the standards “AAMI/ISO TIR10974” established by the Association for the Advancement of Medical Instrumentation (AAMI) of the United States.

Safety limit values for different implants are specified in the implant safety manuals or standards, which may provide guidance for performing safe scans. However, if the safety limit values are too low, it may not be possible to obtain images that meet clinical diagnostic requirements. Moreover, for a patient having a plurality of implants, it is often unclear to physicians how to set appropriate scanning parameters so as to acquire images that meet clinical diagnostic requirements while avoiding safety issues during the scanning process.

### SUMMARY

One aspect of the present invention provides a scanning method based on a magnetic resonance system, the method comprising: for at least one scanning parameter, acquiring a numerical distribution map determined on the basis of the magnetic resonance system; determining an implant-related sensitive region of a subject to be scanned; determining, from the numerical distribution map, a first numerical value corresponding to the sensitive region; acquiring a limit value of the at least one scanning parameter limited by an implant of the subject; adjusting the limit value of the at least one scanning parameter on the basis of the first numerical value; and scanning the subject by using the adjusted limit value of the at least one scanning parameter.

Another aspect of the present invention provides a magnetic resonance scanning system, comprising a magnetic resonance assembly and a controller, the controller being configured to control the magnetic resonance assembly to scan a subject to be scanned and to perform the magnetic resonance scanning method in the above aspect.

### BRIEF DESCRIPTION OF THE DRAWINGS

The described and other features, aspects, and advantages of the present invention will be better understood once the following detailed description has been read with reference to the accompanying drawings. In the accompanying drawings, the same reference signs are used to represent the same components throughout the accompanying drawings, in which:

FIG. 1 shows a block diagram of a magnetic resonance scanning system according to some embodiments;

FIG. 2 is a schematic view of setting safety limit values for scanning parameters in an implant safety manual;

FIG. 3 is an example of a subject to be scanned having a plurality of implants;

FIG. 4 is a flowchart of a scanning method based on a magnetic resonance system according to some embodiments of the present invention;

FIG. 5 shows an example of a body model and of adding an implant identifier to the body model;

FIG. 6 shows an example of edge information of an implant identifier;

FIG. 7 is a flowchart of a scanning method based on a magnetic resonance system according to some other embodiments of the present invention;

FIG. 8 shows an example of a numerical distribution map and a body model;

FIG. 9 is a flowchart of a scanning method based on a magnetic resonance system according to some other embodiments of the present invention;

FIG. 10 is a flowchart of a scanning method based on a magnetic resonance system according to some other embodiments of the present invention;

FIG. 11 is a flowchart of a scanning method based on a magnetic resonance system according to some other embodiments of the present invention; and

FIG. 12 is a block diagram of a magnetic resonance system according to some embodiments of the present invention.

### DETAILED DESCRIPTION

Specific embodiments of the present invention will be described in detail below with reference to the accompanying drawings in order to assist those skilled in the art to understand exactly the subject matter set forth in the present invention. In the following detailed description of the following specific embodiments, the present specification does not describe in detail any known functions or configurations to prevent unnecessary details from affecting the disclosure of the present invention.

Unless otherwise defined, the technical or scientific terms used in the claims and the description should be as they are usually understood by those possessing ordinary skill in the technical field to which they belong. Terms such as “first”, “second”, and similar terms used in the present description and claims do not denote any order, quantity, or importance, but are only intended to distinguish different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections. Furthermore, it should be understood that references to “an embodiment”, “some embodiments” or “embodiments” of the present disclosure are not intended to be construed as excluding the existence of additional implementations that also include the referenced features.

A “module”, “unit”, etc., as described herein may be implemented by using software, hardware, or a combination of software and hardware. For example, according to some aspects of the embodiments of the present invention, the “module” described herein may be implemented as a computer program module or a circuit module.

An “image” described herein may include a displayed image, or may include data that forms the displayed image.

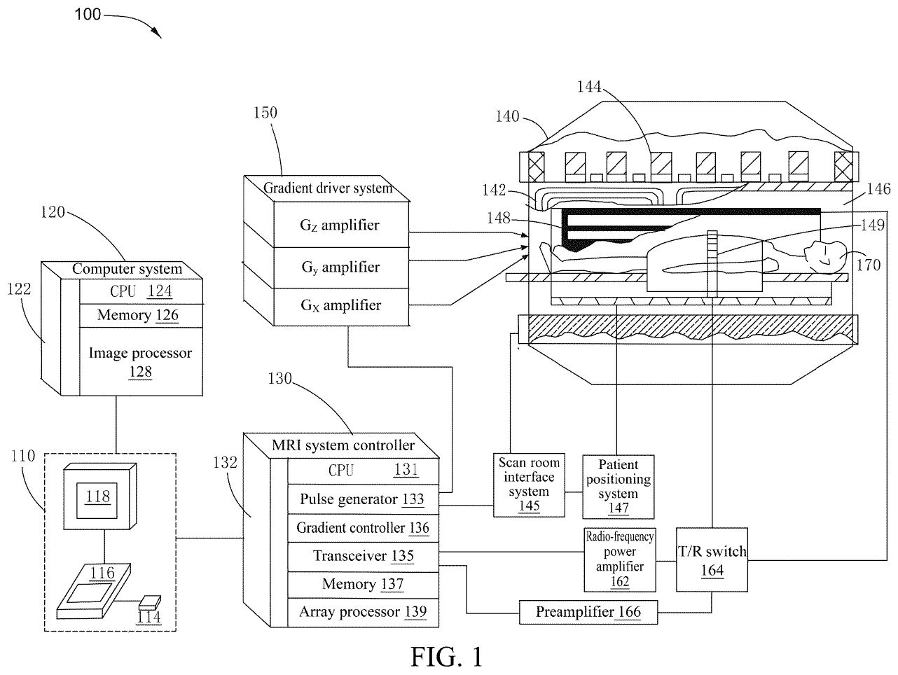

Referring to FIG. 1, an exemplary MRI scanning system 100 according to some embodiments of the present invention is illustrated. An operator workstation 110 is used to control the operation of the MRI system 100, the operator workstation 110 including an input apparatus 114, a control panel 116, and a display 118. The input apparatus 114 may be a joystick, a keyboard, a mouse, a trackball, a touch-activated screen, voice control, or any similar or equivalent input device. The control panel 116 may include a keyboard, a touch-activated screen, voice control, a button, a slider, or any similar or equivalent control device. The operator workstation 110 is coupled to and in communication with a computer system 120, and provides an interface to allow an operator to plan magnetic resonance scanning, display an image and a graphical user interface, perform image processing, and store data and images.

The computer system 120 includes a plurality of modules that communicate with one another by means of an electrical and/or data connection module 122. The connection module 122 may be a wired communication link, an optical fiber communication link, a wireless communication link, and the like. The computer system 120 may include a central processing unit (CPU) 124, a memory 126, and an image processor 128. In some embodiments, the image processor 128 may be replaced with image processing functions run in the CPU 124. The computer system 120 may be connected to an archive media device, a persistent or backup memory, or a network. The computer system 120 may be coupled to and communicates with a separate MRI system controller 130.

The MRI system controller 130 includes a set of modules that communicate with one another by means of an electrical and/or data connection module 132. The connection module 132 may be a direct wired communication link, an optical fiber communication link, a wireless communication link, and the like. In an alternative embodiment, modules of the computer system 120 and the MRI system controller 130 may be implemented on the same computer system or on a plurality of computer systems. The MRI system controller 130 may include a CPU 131, a sequence pulse generator 133 that communicates with the operator workstation 110, a transceiver (or an RF transceiver) 135, a gradient controller 136, a memory 137, and an array processor 139.

A subject 170 undergoing MR scanning may be positioned within a cylindrical imaging volume 146 of a magnetic resonance assembly 140 via a scanning table, a camera (or other auxiliary positioning means), etc., and the subject 170 may have one or more implants in the body thereof. The MRI system controller 130 controls the scanning table to travel in a Z-axis direction of a magnetic resonance system, so as to deliver a predetermined site to be scanned of the subject 170 into the imaging volume 146. The magnetic resonance assembly 140 includes a superconducting magnet having a superconducting coil 144, a radio frequency (RF) coil assembly, and a gradient coil assembly 142. The superconducting coil 144 has a magnet aperture to form the cylindrical imaging volume 146. During operation, the superconducting coil 144 provides a static uniform longitudinal magnetic field B₀ throughout the cylindrical imaging volume 146. The radio-frequency coil assembly may include a body coil 148 and a surface coil 149, and may be used to send and/or receive a radio-frequency signal.

The MRI system controller 130 may receive a command from the operator workstation 110 to indicate a scan sequence to be performed during an MRI scan. The “scan sequence” above refers to a combination of pulses that have specific intensities, shapes, time sequences, and the like and that are applied when a magnetic resonance imaging scan is executed. The pulses may typically include, for example, radio frequency pulses and gradient pulses. A plurality of scan sequences may be pre-stored in the computer system 120, so that a sequence suitable for clinical examination requirements can be indicated by means of the operator workstation. The clinical examination requirements may include, for example, an imaging site, an imaging function, an imaging effect, scanning safety, and the like. The sequence pulse generator 133 of the MRI system controller 130 sends, on the basis of the indicated sequence, an instruction describing the time sequences, intensities, and shapes of the radio frequency pulse and gradient pulse in the sequence so as to operate a system component that executes the sequence.

A radio frequency pulse in the scan sequence sent by the pulse generator 133 may be generated by the transceiver 135, and the radio frequency pulse is amplified by a radio frequency power amplifier 162. The amplified radio frequency pulse is provided to the body coil 148 via a transmit/receive switch (T/R switch) 164, and the RF body coil 148 then immediately provides a transverse magnetic field. As a non-limiting example, a transmitting portion of the transceiver 135, the radio frequency power amplifier 162, the T/R switch 164, and the like constitute at least a portion of a radio frequency transmit link. The transverse magnetic field is substantially perpendicular to B₀ throughout the cylindrical imaging volume 146, and the transverse magnetic field is used to excite stimulated nuclei within the body of the scan subject to generate an MR signal.

The strength of the radio frequency pulse (or radio-frequency field strength) B1 may be set on the basis of different imaging application settings. Typically, the greater the radio-frequency field strength B1, the greater the emitted radio frequency power. A portion of the energy loaded by the radio frequency pulse is released in the form of heat, which is absorbed by the human body, and over time, the energy is deposited at a scan site of the human body, thereby resulting in a local or systemic temperature increase of the human body. The energy that can be absorbed per unit of time and per unit of body weight is defined as SAR (specific absorption rate), and different SAR values are usually set for different sites of the body. When a patient has an implant, more radio-frequency energy may be accumulated at the site of the implant, posing safety risks to the patient's body. Therefore, it may be necessary to set limit values for the radio-frequency field strength B1 and SAR for the implant to avoid safety issues. The limit value of the radio-frequency field strength B1 may be a limitation on any one among a radio-frequency field peak value, a radio-frequency field mean value, and a radio-frequency field root mean square value (B1+rms).

The gradient pulse in the scan sequence sent by the pulse generator 133 may be generated by the gradient controller 136 and acts on a gradient driver 150. The gradient driver 150 includes Gₓ, G_{y}, and G_{z} amplifiers, and the like. Each of the Gₓ, G_{y}, and G_{z} gradient amplifiers is used to excite a corresponding gradient coil (for example, gradient coils respectively arranged along an X-axis, a Y-axis, and a Z-axis of the magnetic resonance system) in the gradient coil assembly 142 so as to generate a gradient magnetic field superimposed on a main magnetic field, forming a magnetic field gradient for spatial encoding of MR signals during MR scanning.

During magnetic resonance scanning, the superimposed gradient field needs to be switched rapidly, so that the magnetic field strength changes over time. The rate at which the magnetic field strength changes over time is the magnetic field change rate (dB/dt), and another parameter related thereto is the maximum gradient slew rate (Max Gradient Slew Rate). Rapid changes in the magnetic field may cause displacement of the implant in the body of the patient and result in a safety issue. Therefore, when an implant exists in the subject to be scanned, the value of the magnetic field change rate may need to be limited.

The pulse generator 133 is coupled to and communicates with a scan room interface system 145, and the scan room interface system 145 can receive signals from various sensors associated with the state of the magnetic resonance assembly 140 and various processors provided in a scan room. The scan room interface system 145 is further coupled to and communicates with a patient positioning system 147, the patient positioning system 147 sending and receiving a signal to control the scanning table to travel so as to transport the patient or the subject 170 to a desired position to perform the MR scan.

As described above, the RF body coil 148 and the RF surface coil 149 may be used to transmit a radio frequency pulse and/or receive MR signals from the scan subject. The MR signals emitted by excited nuclei in the body of the scan subject may be sensed and received by the RF body coil 148 or the RF surface coil 149 and then sent back to a preamplifier 166 by means of the T/R switch 164. The T/R switch 164 may be controlled by a signal from the MRI system controller 130 to electrically connect, during a transmit mode, the radio-frequency power amplifier 162 to the RF body coil 148 and to connect, during a receive mode, the preamplifier 166 to the RF body coil 148. The T/R switch 164 may further enable the RF surface coil 149 to be used in the transmit mode or the receive mode.

In some embodiments, the MR signals sensed and received by the RF body coil 148 or the RF surface coil 149 and amplified by the preamplifier 166 are demodulated, filtered, and digitized in a receiving portion of the transceiver 135, and transmitted as a raw k-space data array to the memory 137 in the MRI system controller 130.

A reconstructed magnetic resonance image may be acquired by transforming/processing the stored raw k-space data. For each image to be reconstructed, the data is rearranged into separate k-space data arrays, and each of the separate k-space data arrays is input into the array processor 139, the array processor being operated to transform the data into an array of image data.

The array processor 139 uses transform methods, most commonly Fourier transform, to create images from the received MR signals. These images are transmitted to the computer system 120 and stored in the memory 126. In response to commands received from the operator workstation 110, the image data may be stored in a long-term memory, or may be further processed by the image processor 128 and transmitted to the operator workstation 110 for presentation on the display 118.

In various embodiments, components of the computer system 120 and MRI system controller 130 may be implemented on the same computer system or on a plurality of computer systems. It should be understood that the MRI system 100 shown in FIG. 1 is intended for illustration. Suitable MRI systems may include more, fewer, and/or different components.

The MRI system controller 130 and the image processor 128 may separately or jointly include a computer processor and a storage medium, the storage medium recording a program for predetermined data processing that is to be executed by the computer processor. For example, the storage medium may store a program used to implement scanning processing (such as a scanning parameter configuration, a scan control procedure, an imaging sequence), image reconstruction, image processing, and the like. Specifically, the storage medium may store a scanning method based on a magnetic resonance system according to any embodiment of the present invention. The described storage medium may include, for example, a ROM, a floppy disk, a hard disk, an optical disk, a magneto-optical disk, a CD-ROM, or a non-volatile memory card.

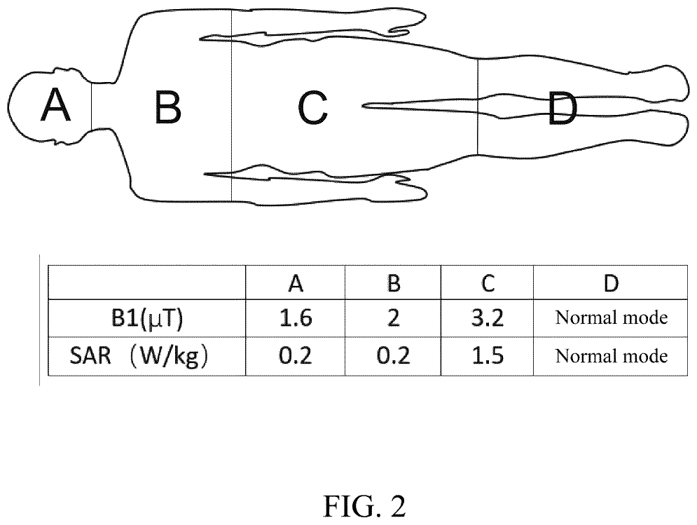

As shown in FIG. 2, in the safety manual of some implants, such as Deep Brain Stimulation (DBS) from Boston Science, Inc., different limit values for the radio-frequency field root mean square value (B1+rms) are set for different body regions A, B, C and D, and different limit values for the SAR are set for the different body regions A, B, C and D. On the basis of the positioning information of the scan subject in the magnetic resonance system, it is recommended to use parameter limit values of region A if the system center (ISO center) is positioned in region A or region A is aligned with the ISO center of the magnetic resonance system. Correspondingly, if region B is aligned with the ISO center, it is recommended to use parameter limit values of region B. For region D, no limit value is set, and scanning may be performed in a normal mode if the ISO center is positioned in region D.

However, the ISO center may be positioned between adjacent regions, causing physicians to be uncertain as to which set of limit values should be selected, which may result in undesirable image quality when the most stringent limit values such as the limit values for region A are used.

There are also some implant safety manuals that specify only stringent parameter limit values without providing more detailed analysis based on the region to be scanned. For example, Biomet Spine Simulator Manual, the safety manual of BIOMET company, specifies a limit value of 250 Gauss/cm for the spatial gradient field strength, a limit value of 20 Tesla/see for the magnetic field change rate, and a SAR limit value of 1.1 Watts/kilogram for a scanning time of 25 minutes. Stringent limit values may make some scan sequences unusable or result in undesirable image quality. When a region of interest to be scanned is far away from the implant, the use of a higher limit value may better balance image quality and safety. However, the physician may adopt a more conservative parameter setting due to the inability to determine how to set the limit value.

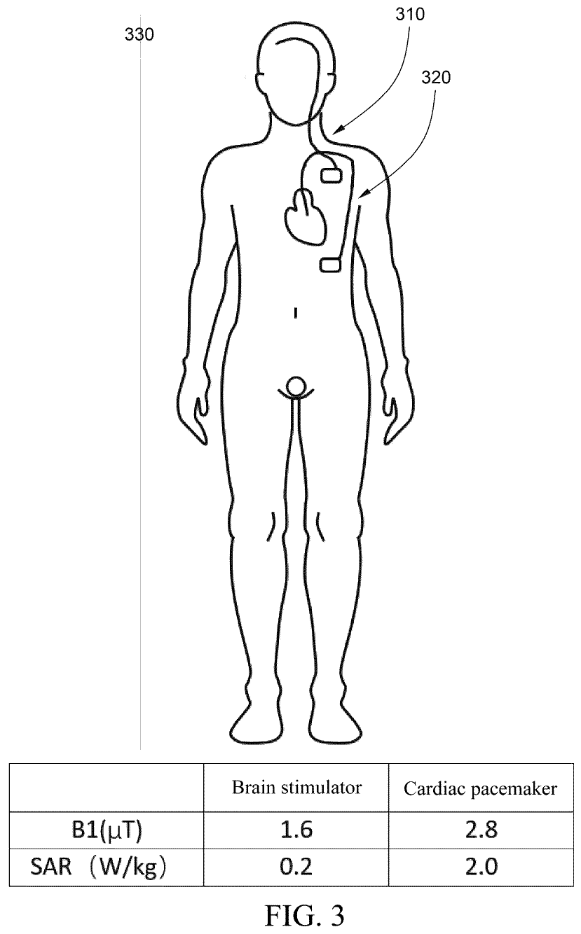

When the subject to be scanned has a plurality of implants, selecting the most stringent implant safety limit value can maximally avoid safety issues, but this may result in the inability to perform normal scanning. FIG. 3 shows an example of a subject to be scanned having a plurality of implants, including a brain stimulator 310 and a cardiac pacemaker 320. According to corresponding safety manuals, the limit values for the brain stimulator 310 are: SAR less than or equal to 0.2 W/kg (Watts/kilogram) and B1rms (B1) less than or equal to 1.6 μT (microtesla), and the limit values for the cardiac pacemaker 320 are: SAR less than or equal to 2 W/kg and B1rms (B1) less than or equal to 2.8 μT. For safety reasons, safety limit values for the brain stimulator 310 may be selected during a magnetic resonance scan, but this may cause image issues.

The inventors of the present application have found that implant manufacturers or standards associations typically analyze the safety of scanning parameters on the basis of theoretical values, a simple magnetic resonance simulation environment, a single or separate gradient field or radio-frequency field environment, or comprehensive parameters of magnetic resonance products from multiple manufacturers. However, some parameter limit values may have room for adjustment due to differences of actual magnetic resonance products from the above analysis.

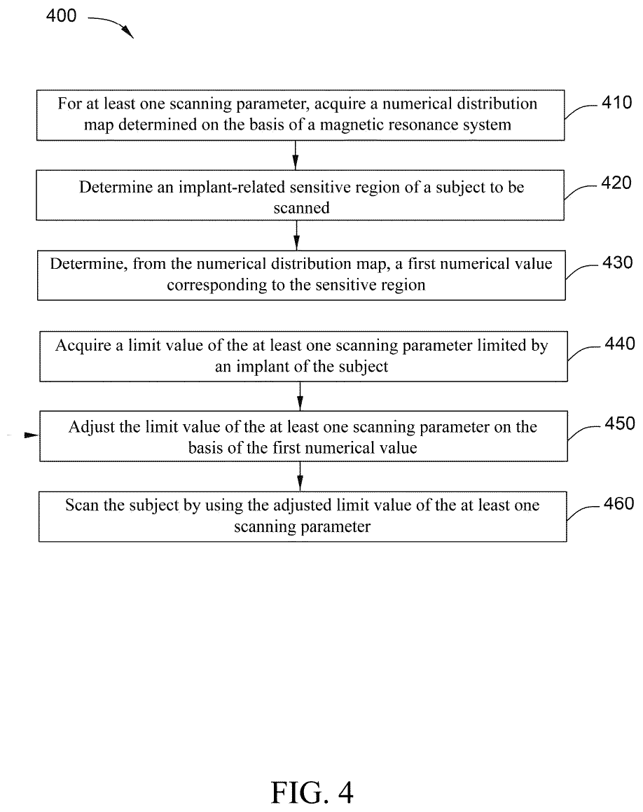

FIG. 4 shows a flowchart 400 of a scanning method based on a magnetic resonance system according to an embodiment of the present invention. In step 410, a numerical distribution map determined on the basis of the magnetic resonance system is acquired for at least one scanning parameter. The at least one scanning parameter may be a parameter whose range of values is limited by an implant, and may include, for example, one or more among radio-frequency field strength B1 (e.g., B1+rms), SAR (which may include the SAR value for the whole body, SAR for the head, and SAR for other body sites), spatial gradient field strength (Spatial Gradient), maximum gradient slew rate (Max Slew Rate), and magnetic field change rate (dB/dt), etc. The numerical distribution map of the at least one scanning parameter is determined on the basis of the magnetic resonance system; for example, the map may be obtained by measuring, simulating, or monitoring a magnetic resonance system that is currently performing a magnetic resonance scan on a scan subject, wherein the numerical values of the scanning parameter may be distributed in the magnetic field space coordinates of the magnetic resonance system to form a numerical array or a color temperature map. The numerical distribution map may be pre-stored in the magnetic resonance system, or acquired in real-time when performing the scanning method according to the embodiment of the present invention. The numerical distribution map may include, for example, a radio-frequency field map, a SAR distribution map, a magnetic field change rate map for different gradient axes, a spatial gradient field map, etc.

In step 420, an implant-related sensitive region of a subject to be scanned is determined. The sensitive region may be a region where an implant is located, or a region where safety limit values are relatively low.

In some embodiments, the sensitive region may be determined with reference to an implant safety manual. For example, when a plurality of limit value regions are specified in an implant safety manual (e.g., as shown in FIG. 2), the sensitive region may be the region with the most stringent limit values (for example, region A). In another example, when parameter limit values are respectively set for a plurality of implants in a plurality of implant safety manuals, the sensitive region may be the region with the most stringent limit values (for example, a region 330 where the brain stimulator 310 is located in FIG. 3) among regions where the plurality of implants are located. In yet another example, if the implant safety manual does not include divided limit value regions and only parameter limit values are specified therein, a region where a corresponding implant is located in the subject to be scanned may be determined as the sensitive region.

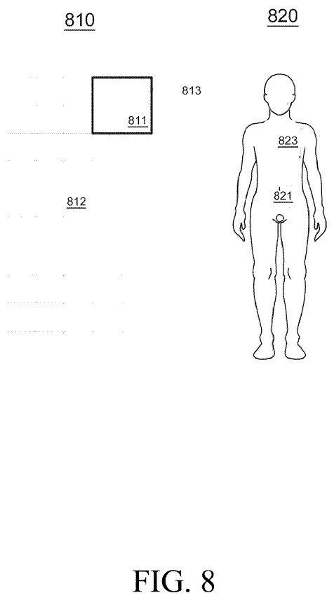

In step 430, a first numerical value corresponding to the sensitive region is determined from the numerical distribution map. As described above, the numerical values of the scanning parameters in the numerical distribution map may be distributed in the magnetic field space coordinates of the magnetic resonance system, and thus can correspond to spatial position information of the magnetic resonance system. When undergoing a magnetic resonance scan, the subject is positioned in the magnetic field space of the magnetic resonance. Therefore, the position information of the subject to be scanned and the numerical distribution map can be matched by means of the spatial coordinates of the magnetic resonance system, so that a numerical value corresponding to the sensitive region of the subject can be determined from the numerical distribution map. Step 430 will be described in detail below with reference to FIG. 8.

In step 440, a limit value of the at least one scanning parameter limited by an implant of the subject is acquired, and the limit value acquired in this step may be a unique limit value for the scanning parameter or the “most stringent limit value” among a plurality of limit values, such as the limit value corresponding to region A in FIG. 2 or the limit value corresponding to the brain stimulator 310 in FIG. 3.

In step 450, the limit value of the at least one scanning parameter is adjusted on the basis of the first numerical value.

In step 460, the subject is scanned by using the adjusted limit value.

Generally, the radio-frequency field and the main magnetic field of a magnetic resonance system are not completely uniform, but vary with respect to the system center (ISO center). When system parameters are set, the above-mentioned safety limit values are also applied to the system center instead of the region where an implant is located, and when the safety limit values are determined, it is also generally assumed that the sensitive region is located at the system center. However, during actual scanning, the region where an implant is located or the above-mentioned sensitive region may be far away from the system center. In the embodiment of the present invention, an actual parameter value distribution map of the magnetic resonance system is acquired, and a numerical value corresponding to an actual sensitive region is determined therefrom to adjust the safety limit value, so that a more accurate safety limit value can be acquired.

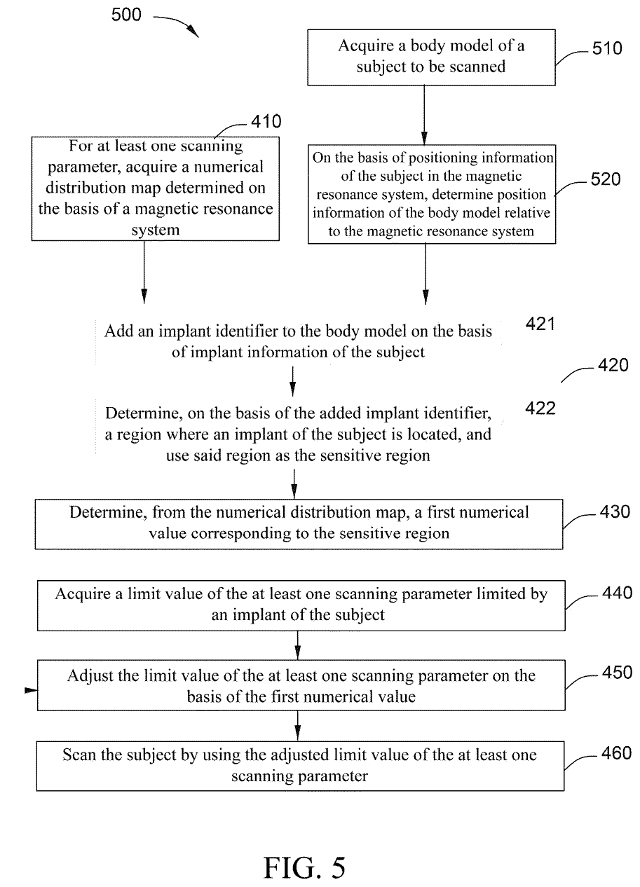

FIG. 5 shows a flowchart 500 of a scanning method based on a magnetic resonance system according to some other embodiments of the present invention, in which steps 510 and 520 are further included. In step 510, a body model of a subject to be scanned is acquired. In step 520, position information of the body model relative to the magnetic resonance system is determined on the basis of positioning information of the subject in the magnetic resonance system. Specifically, a corresponding site to be scanned can be determined from the body model, and the center thereof can be aligned with the system center, so that the position information of the body model relative to the magnetic resonance system is determined, said position information also corresponding to the numerical distribution map of the magnetic resonance system. As shown in FIG. 5, step 420 may specifically include: determining a sensitive region from the body model.

Optionally, the sensitive region includes a region where an implant of the subject is located. When there are a plurality of implants, the sensitive region includes a region where the implant having the most stringent limit value for the at least one scanning parameter is located.

In step 420, the sensitive region where an implant is located can be determined by adding an implant identifier to the body model, and said step may specifically include steps 421 and 422. In step 421, an implant identifier is added to the body model on the basis of implant information of the subject. In step 422, a region where an implant of the subject is located is determined on the basis of the added implant identifier, wherein the region where an implant is located serves as the sensitive region.

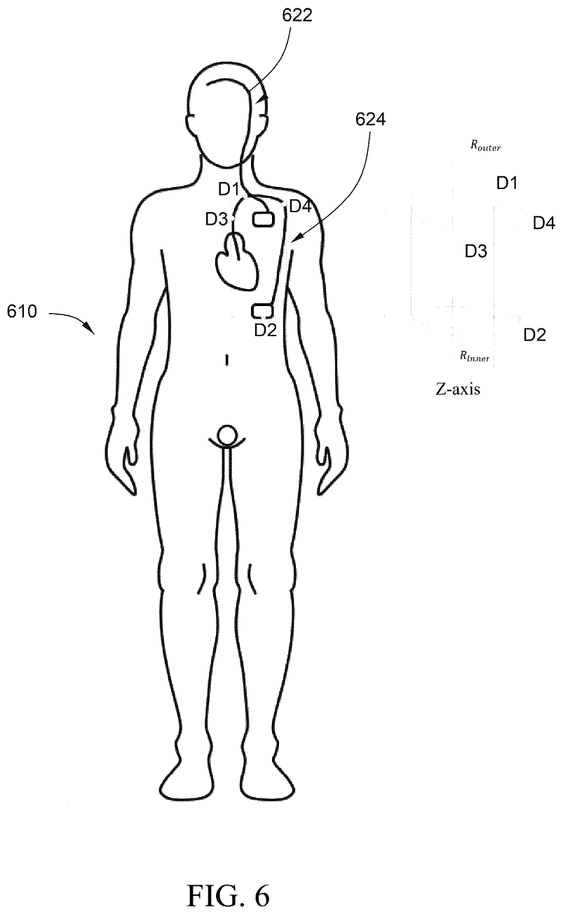

How to acquire the body model and determine the sensitive region in the body model will be described in detail below with reference to FIG. 6. FIG. 6 exemplifies a body model 610. In some embodiments, the body model 610 may be obtained on the basis of an optical image of the subject to be scanned. For example, an optical camera may be used to photograph the subject to be scanned positioned on a scanning table, to obtain a two-dimensional or three-dimensional optical image of the subject. The optical image may be used directly as the body model of the subject to be scanned or may be used as the body model after appropriate calibration. Alternatively, the optical image may be used to perform alignment on a pre-stored standard body model to obtain a personalized body model adapted to the body size of the subject to be scanned.

The body model may be a three-dimensional model having a human-like structure. For example, the body model may include, but is not limited to, the torso, limbs, head, brain, heart, liver, spleen, stomach, bone, etc., to facilitate the addition of implant identifiers at more accurate positions. The body model may have a more simplified structure, e.g., may include only an outer contour, and a user may add implant identifiers in the simplified body model on the basis of their own knowledge of human anatomy. The body model may be obtained after being matched with the subject to be scanned, and “matching” may include being consistent with or similar to the subject to be scanned in terms of height, weight, body proportions, gender, age, etc. In an example of the present invention, implant identifiers may be added to the body model 610 by means of a graphical user interface.

Different types and models of implants may have different shapes, structures, and sizes. Implant identifiers representing different implants may be pre-stored in the magnetic resonance system, and can have similar shapes and structures to those of actual implant products, so that an operator can quickly select an appropriate implant identifier according to implant information of the subject (e.g. one or more of implant type, model, size, body site, etc.) and add the same to the body model. As shown in FIG. 6, which shows implant identifiers 622 and 624 added to the body model 610, the implant identifier 622 represents, for example, a brain stimulator, the implant identifier 624 represents, for example, a cardiac pacemaker, and the implant identifiers 622 and 624 may include one or more of electrodes, leads, cartridges, etc.

One of the regions in the body model where implant identifiers are located (for example, the region corresponding to the most stringent safety limit value) may be used as the sensitive region. In some embodiments, the region where an implant is located may be automatically determined on the basis of the added implant identifier and the position information of the body model. For example, the region where an implant is located may be determined on the basis of edge position information of the implant identifier, and the region may be, for example, an annular cylindrical region defined by the edge information and radii. As shown in FIG. 6, the edge information may include, for example, Z-axis coordinate information (Zₛₜₐᵣₜ and Z_{end}) of two ends (e.g., D1 and D2) that are farthest from each other in the Z-axis direction on the edge of the implant identifier, and radius information (Rᵢₙₙₑᵣ and Rₒᵤₜₑᵣ) of two ends (for example, D3 and D4) that are farthest from each other in the X-axis (and/or Y-axis) direction on the edge of the implant identifier, wherein radius information Rᵢₙₙₑᵣ and Rₒᵤₜₑᵣ are coordinate information of end points D3 and D4 with respect to the system center, respectively, and represent the distances from end points D3 and D4 to the Z center axis (passing through the system center), respectively.

In the embodiments of the present invention, the region where an implant is located may also be determined by other computer-aided means on the basis of the added implant identifier.

In the embodiments of the present invention, the sensitive region can be more accurately determined by operating the body model, and a more accurate adjustment value for the safety limit value can be further obtained. However, after the positioning information of the subject in the magnetic resonance system is determined on the basis of the site to be scanned, numerical regions corresponding to the sensitive region may also be directly estimated in the radio-frequency field map and the SAR map of the magnetic resonance system, respectively, thereby eliminating the need to operate the body model, and simplifying the scanning process.

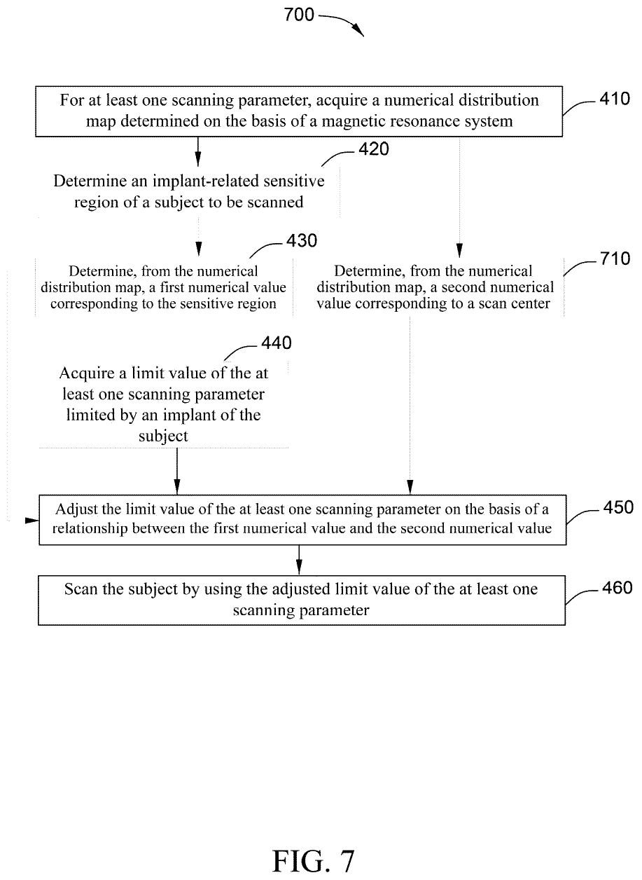

FIG. 7 shows a flowchart 700 of a scanning method based on a magnetic resonance system according to some other embodiments of the present invention, the method including the steps shown in FIG. 4, and further including step 710: determining, from the numerical distribution map, a second numerical value corresponding to a scan center. The scan center may be a position that coincides with the system center. More specifically, in step 450, the limit value of the at least one scanning parameter may be adjusted on the basis of a relationship between the first numerical value and the second numerical value. Further, the relationship may be a proportional relationship.

As described above, the position information of the body model relative to the magnetic resonance system may be determined on the basis of positioning information of the subject in the magnetic resonance system. In an embodiment of the present invention, the following step may be further included: on the basis of predetermined positioning information of the subject in the magnetic resonance system, acquiring a position correspondence between the body model and the numerical distribution map; wherein the first numerical value is determined on the basis of the position correspondence. Description will be made below with reference to the example of FIG. 8.

FIG. 8 shows an example of a numerical distribution map 810 and a body model 820, wherein the numerical distribution map 810 uses a grid to represent numerical values distributed on coordinate axes, for example, including a first numerical value 811 and a second numerical value 812. The second numerical value 812 is a parameter value at the system center, that is, said numerical value has zero coordinates. A region of interest 821, which corresponds to a site to be scanned of the subject, is determined from the body model 820. The center of the region of interest 821 is the scan center, and the coordinate position thereof is defined as the coordinate center of the magnetic resonance system, i.e., the system center, such as the zero coordinates. A sensitive region 823, which may be, for example, a region where a cardiac pacemaker is located, is also determined from the body model 820. A safety limit value for magnetic resonance scanning is specified in the safety manual of the cardiac pacemaker. The numerical distribution map 810 has a numerical region 813 whose coordinate position corresponds to the coordinate position of the sensitive region 823. The first numerical value is a numerical value in the numerical region 811. In some embodiments, to achieve scanning safety, a maximum numerical value is selected from the numerical region 811 as the first numerical value. However, the first numerical value may be determined in other ways, such as determining the first numerical value 811 by performing a mathematical operation on the basis of all or part of the numerical values in the numerical region 813.

As described above, the main magnetic field, the radio-frequency field, and the like of the magnetic resonance system are not completely uniform, resulting in relevant parameter values in the spatial coordinates being not completely the same. Therefore, the first numerical value and the second numerical value may be different. Further, the first numerical value and the second numerical value may change in equal proportion. For example, when the second numerical value changes, the first numerical value changes along with the second numerical value in the same proportion. Therefore, in step 450, the safety limit value (or the current limit value) in the safety manual may be specifically adjusted on the basis of the proportional relationship.

More specifically, the adjusted limit value of the at least one scanning parameter is the product of the ratio between the second numerical value and the first numerical value, and the limit value before the adjustment. For example, the safety limit value in the manual may be adjusted using the following equation (1):

$${L}_{s c an - limit} = {L}_{m a n u a l - limit} \times \frac{{L}_{2}}{{L}_{1}} ; \qquad ( 1 )$$

where L_{scan-limit} is the adjusted limit value of the at least one scanning parameter, Lₘₐₙᵤₐₗ₋ₗᵢₘᵢₜ is the limit value configured for the at least one scanning parameter in the safety manual, L₁ is the first numerical value, and L2 is the second numerical value.

On the basis of the numerical distribution map of the magnetic resonance system, the numerical value at the system center is usually the maximum numerical value, and the numerical values at other positions become smaller as the distance from the system center becomes larger. Therefore, the above configuration enables stringent safety limit values in the manual to be adjusted to have a larger limit value range, providing image quality that can better meet clinical diagnosis requirements while achieving safe scanning.

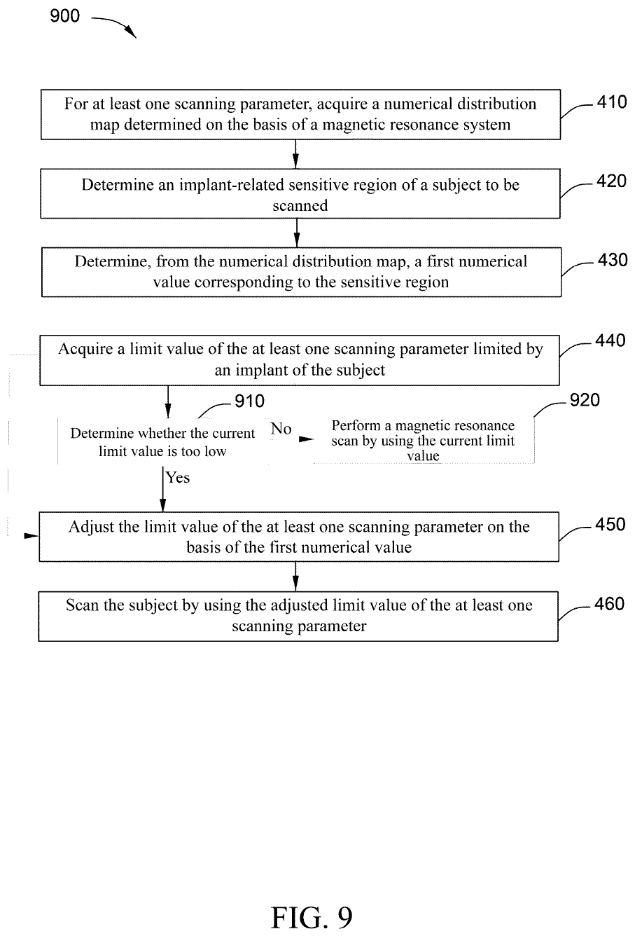

FIG. 9 shows a flowchart 900 of a scanning method based on a magnetic resonance system according to another embodiment of the present invention, the method including the following optional step 910: determining whether the current limit value (such as values specified in the safety manual) is too low; if so, adjusting the current limit value, that is, executing step 450; and if not, executing step 920: performing a magnetic resonance scan by using the current limit value. In step 910, whether the limit value is too low may be determined by using the current safety limit value to perform simulation scanning, quality assessment, or the like, or the operator may be asked by means of human-machine interaction.

The safety limit values shown in FIG. 2 will be used as an example for explanation. According to the safety limit values in the safety manual for the respective regions in FIG. 2, the most stringent safety limit values for both the scanning parameters “radio-frequency field strength” and “SAR” are specified for region A, wherein the B1 limit value is 1.6 μT (microtesla) and the SAR limit value is 0.2 W/kg. According to an embodiment of the present invention, the safety limit values of the two parameters are adjusted by the following steps.

Step 1: determining a body region of the subject corresponding to region A as the sensitive region.

Step 2: acquiring a body model of the subject, and determining position information of the body model relative to the magnetic resonance system.

Step 3: determining the sensitive region from the body model, for example, drawing a region matching region A directly at the head position of the model, or adding a corresponding implant identifier (e.g. a brain stimulator) to determine the sensitive region.

Step 4: determining numerical regions corresponding to the sensitive region in the radio-frequency field map and the SAR distribution map of the magnetic resonance system, respectively, and setting the maximum values thereof as first numerical values (for example, 2.0 μT and 1.0 W/kg, respectively).

Step 5: determining second numerical values (e.g., 3.0 μT and 1.5 W/kg, respectively) corresponding to the scan center from the radio-frequency field map and the SAR distribution map, respectively.

Step 6: substituting the first numerical values and the second numerical values into the following equations (2) and (3), respectively, to obtain adjusted limit values,

$$B {1}_{s c an - limit} = B {1}_{m a n u al - limit} \times \frac{B {1}_{2}}{B {1}_{1}} ; \qquad ( 2 )$$

$$SA {R}_{s c a n - limit} = S A {R}_{m a n u a l - limit} \times \frac{S A {R}_{2}}{S A {R}_{1}} \qquad ( 3 )$$

where B1ₘₐₙᵤₐₗ₋ₗᵢₘᵢₜ is 1.6 μT, B1₂ is 3.0 μT, B1₁ is 2.0 μT, the adjusted B1 limit value B1_{scan-limit} is 2.4 μT; SARₘₐₙᵤₐₗ₋ₗᵢₘᵢₜ is 0.2 W/kg, SAR₂ is 1.5 W/kg, SAR₁ is 1.0 W/kg, and the adjusted SAR limit value SAR_{scan-limit} is 0.3 W/kg.

Optionally, the at least one scanning parameter described above may further include a magnetic field change rate or a maximum gradient slew rate, and the numerical distribution map may further include a magnetic field change rate distribution map of the magnetic resonance system. Specifically, the magnetic field change rate distribution map may also be numerical values distributed in the spatial coordinates of the magnetic resonance system.

Further, the numerical distribution map may include a magnetic field change rate distribution map of one or more gradient axes of the magnetic resonance system. As described above, the magnetic resonance system may include X-axis, Y-axis, Z-axis gradient coils, which can be turned on individually to generate gradient fields in respective directions thereof, or turned on simultaneously to generate an equivalent gradient field. Therefore, in the embodiment of the present invention, at least one among an X-axis dB/dt (magnetic field change rate) map, a Y-axis dB/dt map, a Z-axis dB/dt map, and a spatial (or equivalent) dB/dt map formed by the three axes X, Y and Z can be used to adjust the safety limit value of the magnetic field change rate.

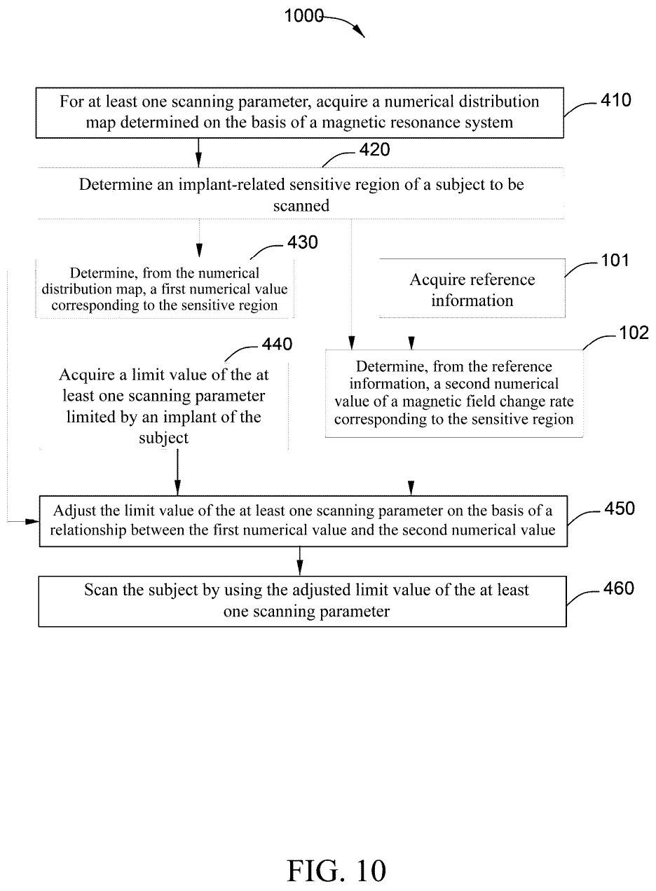

FIG. 10 shows a flowchart 1000 of a scanning method based on a magnetic resonance system according to another embodiment of the present invention, the method including the steps shown in FIG. 4, and further including steps 101 and 102. In step 101, reference information is acquired. In step 102, a second numerical value of a magnetic field change rate corresponding to the sensitive region is determined from the reference information.

The reference information includes externally defined magnetic field change rate distribution information of one or more gradient axes. The magnetic field change rate distribution information includes a magnetic field change rate in a defined spatial coordinate system, the defined spatial coordinate system corresponding to a magnetic field spatial coordinate system of the magnetic resonance system. Table (1) below shows an example of the externally defined magnetic field change speed distribution information. The information is defined by the standards association, specifically, the standards manual “AAMI/ISO TIR10974” from the AAMI, which is cited in the present embodiment only as an example for description, and reference information with other forms, contents, or sources may also be applied to the example of the present embodiment.

TABLE 1

| Radial | dB [symbol] /dt, | | |
|---|---|---|---|
| distance | dB [symbol] /dt [symbol] | dB [symbol] /dt | dB [symbol] /dt |
| cm | T/s | T/s | T/s |
| 5 | 58.0 | 65.7 | 101.6 |
| 6 | [symbol] | 66.5 | 102.9 |
| 7 | 59.7 | 70.1 | 103.7 |
| 8 | 61.0 | 73.4 | 105.3 |
| 9 | 62.4 | 75.6 | 106.7 |
| 10 | 63.7 | 79.3 | 108.7 |
| 11 | 65.2 | 81.7 | 110.5 |
| 12 | 66.9 | 84.2 | 112.4 |
| 13 | [symbol] | 88.7 | 115.8 |
| 14 | 71.3 | 91.9 | 118.5 |
| 15 | [symbol] | 96.7 | 122.3 |
| 16 | 76.0 | 100.1 | 125.3 |
| 17 | [symbol] | 105.9 | 130.2 |
| 18 | 81.9 | 109.6 | 133.5 |
| 19 | 86.2 | 114.0 | 137.7 |
| 20 | 89.8 | 120.4 | 145.4 |
| 21 | 93.8 | 125.2 | 148.1 |
| 22 | 98.3 | 132.7 | 155.2 |
| 23 | 103.0 | 137.9 | 160.4 |
| 24 | 108.2 | 144.1 | 166.8 |
| 25 | 115.7 | 153.3 | 176.0 |
| 26 | 122.4 | 159.2 | 182.3 |
| 27 | 129.4 | 169.3 | 192.8 |
| 28 | 137.2 | 176.7 | 200.9 |
| 29 | 146.0 | [symbol] | 214.1 |
| 30 | [symbol] | 196.8 | 223.5 |

[symbol]

indicates data missing or illegible when filed

As shown in Table (1) above, in the magnetic field change rate distribution information, position information (radial distance) is defined, which represents X-axis dB/dt (dB_{X}/dt), Y-axis dB/dt (dB_{Y}/dt), Z-axis dB/dt (dB_{Z}/dt) and space dB/dt (dBₘ/dt) on a cylindrical surface with a radius of 5-30 cm and the Z-axis as the center, wherein the distribution values of X-axis dB/dt (dB_{X}/dt) and Y-axis dB/dt (dB_{Y}/dt) in the spatial coordinates are the same, and on the basis of the distribution information, it is considered that the distribution values on the same cylindrical surface are all uniform. For example, the distribution values of X-axis dB/dt on the cylindrical surface with a radius (or radial distance) of 5 cm are all 58.0. Moreover, in the standards, safety limit values are configured for the magnetic field change rate or the maximum gradient slew rate at least on the basis of part of the information. In the magnetic field change rate distribution map obtained on the basis of the magnetic resonance system, the distribution values on the same cylindrical surface may be different. For example, X-axis dB/dt on the cylindrical surface having a radius (or radial distance) of 5 cm may change as the spatial position changes, that is, X-axis dB/dt on the surface may have a plurality of values.

In an embodiment of the present invention, the safety limit value of at least one of the magnetic field change rate and the maximum gradient slew rate is further adjusted in combination with the reference information and the numerical distribution map (the magnetic field change rate distribution map) of the magnetic resonance system itself.

Further, the numerical distribution map of the magnetic resonance system may include a magnetic field change rate distribution map of one or more gradient axes (e.g., an X axis, a Y axis, a Z axis, and a spatial axis formed by the three axes X/Y/Z) of the magnetic resonance system, wherein the numerical distribution map of each gradient axis includes a numerical value corresponding to the sensitive region described above, i.e. the first numerical value.

In step 450, the limit value of the at least one scanning parameter may be adjusted on the basis of a relationship between the first numerical value and the second numerical value. More specifically, the relationship is a proportional relationship between the second numerical value and the first numerical value. For example, equation (1) may be used to adjust the magnetic field change rate or the maximum gradient slew rate.

In an embodiment of the present invention, the magnetic field change rate or the maximum gradient slew rate of all or part of the gradient axes may be adjusted by using only the magnetic field change rate distribution map on one gradient axis and the reference information on the corresponding gradient axis. For example, the first numerical value used in equation (1) may come from a magnetic field change rate distribution map of an equivalent axis, and the second numerical value may come from numerical values on the equivalent axis in the reference information.

However, optionally, to further improve the accuracy of limit value adjustments, the magnetic field change rate or the maximum gradient slew rate of all or part of the gradient axes may also be adjusted by simultaneously using the magnetic field change rate distribution maps and the reference information on a plurality of axes.

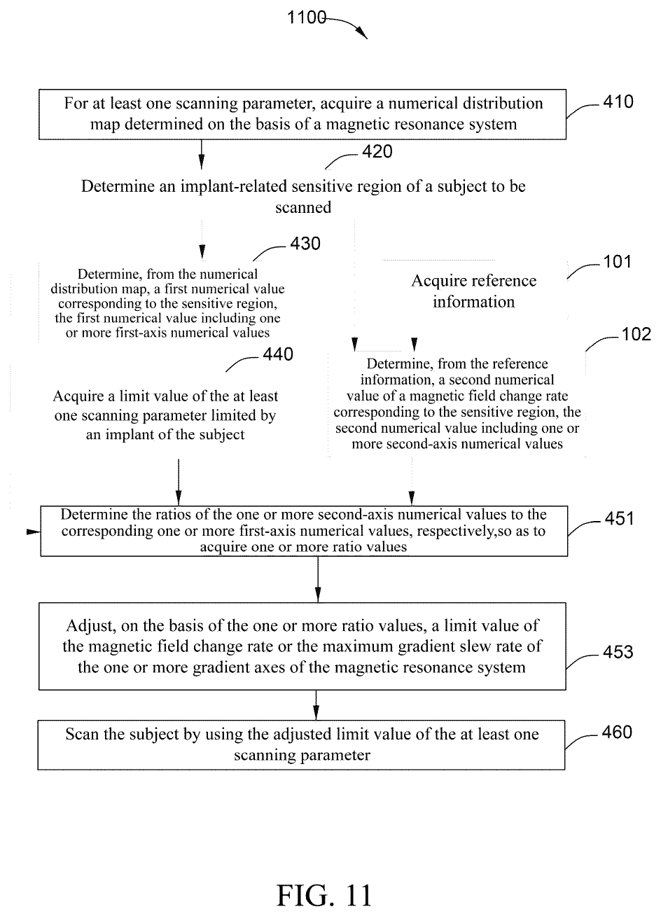

FIG. 11 shows a flowchart 1100 of a scanning method based on a magnetic resonance system according to another embodiment of the present invention, wherein step 450 further includes steps 451 and 453. As described above, both the numerical value map and the external reference information may include the distribution of numerical values (magnetic field change rate) of one or more gradient axes, and thus the first numerical value may include one or more first-axis numerical values, and each first-axis numerical value is: a maximum numerical value determined from a numerical region of a magnetic field change rate distribution map of one gradient axis of the magnetic resonance system, said numerical region corresponding to the sensitive region.

For example, in the magnetic field change rate distribution map of the X-axis, a region corresponding to the sensitive region has a plurality of numerical values thereon, and the maximum value among them is selected as the first-axis numerical value B_{X-implant} of the X-axis. Correspondingly, the first-axis numerical value of the Y axis, the first-axis numerical value of the Z axis, and the first-axis numerical value of the equivalent axis can be determined from the magnetic field change rate distribution maps of the Y axis, the Z axis, and the equivalent axis, respectively, and are denoted as B_{Y-implant}, B_{Z-implant} and B_{M-implant}, respectively.

Similarly, the second numerical value may include a plurality of second-axis numerical values B_{X}, B_{Y}, B_{Z} and B_{M} corresponding to the sensitive region, the second-axis numerical values being determined from the defined magnetic field change rate distribution information of the one or more gradient axes, respectively. For example, if the region corresponding to the sensitive region in Table (1) above is a region with a radial distance of 10 cm, then the second-axis numerical values B_{X}, B_{Y}, B_{Z} and B_{M} are 63.7, 63.7, 79.3, and 108.7, respectively.

Then, in step 451, the ratios of the one or more second-axis numerical values to the corresponding one or more first-axis numerical values are respectively determined to acquire one or more ratio values, for example,

$$α = \frac{{B}_{X - implant}}{{B}_{X}} , β = \frac{{B}_{Y - implant}}{{B}_{Y}} , γ = \frac{{B}_{Z - implant}}{{B}_{Z}} , and δ = \frac{{B}_{M - implant}}{{B}_{M}} .$$

In step 452, the limit value of the magnetic field change rate or the maximum gradient slew rate of the one or more gradient axes of the magnetic resonance system is adjusted on the basis of the one or more ratio values. For example, parameter limit values for a plurality of gradient axes may be adjusted by using one ratio value, or parameter limit values for a plurality of corresponding gradient axes may be adjusted by using a plurality of ratio values.

Specifically, in an embodiment, step 452 may further include the following steps: determining a minimum value among the one or more ratio values; and adjusting, on the basis of the minimum value, the limit value of the magnetic field change rate of the one or gradient axes of the magnetic resonance system.

For example, the ratio value a may be a minimum value that can be used to adjust the limit value of the magnetic field change rate for all or part of the gradient axes of the magnetic resonance system.

Specifically, the adjusted limit value is the product of the minimum value among the plurality of ratio values and the limit value before the adjustment. For example, the magnetic field change rate and the maximum gradient slew rate of each gradient axis may be adjusted on the basis of the following equations (4) and (5), respectively:

$$d B / {dt}_{limit} = Min ( α , β , γ , δ ) \times d B / {dt}_{i mplant} ; \qquad ( 4 )$$

$$Max\_Slew {\_Rate}_{s c a n} = Min ( α , β , γ , δ ) \times Max\_Slew {\_Rate}_{i mplant} ; \qquad ( 5 )$$

where dB/dtₗᵢₘᵢₜ is the adjusted limit value of the magnetic field change rate, Min (α, β, γ, δ) is the minimum value among the plurality of ratio values α, β, γ, δ, dB/dtᵢₘₚₗₐₙₜ is the current limit value of the magnetic field change rate, such as a safety limit value defined in safety manuals or standards; Max_Slew_Rate_{scan} is the adjusted limit value of the maximum gradient slew rate, and Max_Slew_Rateᵢₘₚₗₐₙₜ is the current limit value of the maximum gradient slew rate, such as a safety limit value defined in safety manuals or standards.

The magnetic field change rate or the maximum gradient slew rate is adjusted by using the minimum value among the ratios corresponding to a plurality of axes, which can be applied to the limit value adjustments of all gradient axes, thereby largely ensuring scanning safety and simplifying the parameter setting process. However, in other embodiments, it is also possible to adjust, on the basis of each ratio value, the limit value of a parameter (e.g., the magnetic field change rate or the maximum gradient slew rate) of the corresponding axis, that is, the parameter limit values of different axes are adjusted according to respective ratios thereof. For example, the magnetic field change rate in the X-axis can be adjusted using equations (6), (7), (8), and (9), respectively:

$$d {B}_{x} / {dt}_{limit} = α \times d {B}_{X} / {dt}_{i mplant} ; \qquad ( 6 )$$

$$d {B}_{y} / {dt}_{limit} = β \times d {B}_{y} / {dt}_{i mplant} ; \qquad ( 7 )$$

$$d {B}_{z} / {dt}_{limit} = γ \times d {B}_{z} / {dt}_{i mplant} ; \qquad ( 8 )$$

$$d {B}_{m} / {dt}_{limit} = δ \times d {B}_{m} / {dt}_{i mplant} ; \qquad ( 9 )$$

where dBₓ/dtₗᵢₘᵢₜ, dB_{y}/dtₗᵢₘᵢₜ, dB_{z}/dtₗᵢₘᵢₜ and dBₘ/dtₗᵢₘᵢₜ are the adjusted limit value of dB/dt of the X axis, the adjusted limit value of dB/dt of the Y axis, the adjusted limit value of dB/dt of the Z axis, and the adjusted limit value of dB/dt of the equivalent axis, respectively, and dBₓ/dtᵢₘₚₗₐₙₜ, dB_{y}/dtᵢₘₚₗₐₙₜ, dB_{z}/dtᵢₘₚₗₐₙₜ and dBₘ/dtᵢₘₚₗₐₙₜ are the current limit value of dB/dt of the X-axis, the current limit value of dB/dt of the Y-axis, the current limit value of dB/dt of the Z-axis, and the current limit value of dB/dt of the equivalent axis, respectively.

Therefore, step 452 may optionally include: adjusting, on the basis of the one or more ratio values, the limit value of the magnetic field change rate or the maximum gradient slew rate of one or more corresponding gradient axes of the magnetic resonance system, respectively. For example, when the above parameters of all or only some of the gradient axes need to be adjusted, the adjustment can be made on the basis of corresponding ratio values without the need to determine the minimum ratio value.

In this way, limit values can be adjusted for the parameters of different axes, respectively, thereby obtaining more accurate scanning parameters.

In an embodiment of the present invention, a cylindrical surface close to the edge of the sensitive region in the cylindrical gradient space is determined as the region corresponding to the sensitive region. For example, as shown in FIG. 6, the sensitive region may be defined as an annular cylinder with Z-axis coordinate information (Zₛₜₐᵣₜ and Zₛₜₐᵣₜ) and radius information (Rᵢₙₙₑᵣ and Rₒᵤₜₑᵣ), and the gradient spatial position of the magnetic resonance system may be described as a plurality of concentric cylinders with varying radii that use the Z axis (passing through the system center) as the center. The region corresponding to the sensitive region is a region corresponding to the outer surface of the circular cylinders.

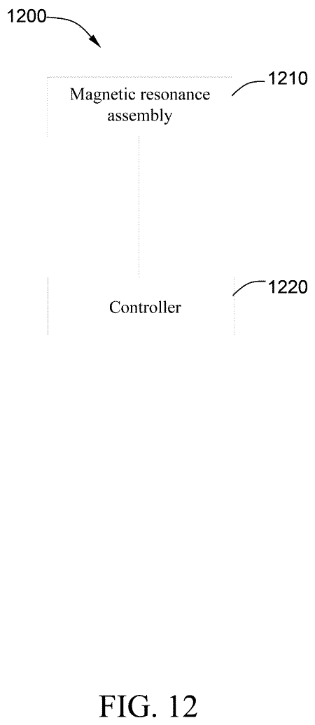

As shown in FIG. 12, some embodiments of the present invention may further provide a magnetic resonance system 1200, including a magnetic resonance assembly 1210 and a controller 1220, wherein the controller 1220 is configured to control the magnetic resonance assembly 1210 to scan a subject to be scanned (such as a subject to be scanned 16 having an implant (not shown in the figure)). The controller is further configured to perform the magnetic resonance scanning method according to any one of the above embodiments.

The controller 1220 may separately communicate with at least a part of the computer system 120 and the MR system controller 130 in FIG. 1, or include at least a part of the computer system 120 and the MR system controller 130, or be integrated with at least a part of the computer system 120 and the MR system controller 130. The magnetic resonance assembly 1210 may further include part or all of the magnetic resonance assembly 140 in FIG. 1.

In the embodiment of the present invention, the limit values of scanning parameters that can better meet clinical diagnostic requirements are determined on the basis of the parameters of the magnetic resonance system itself (e.g. the numerical distribution maps of the parameters), the subject to be scanned (e.g. the determined sensitive region), the current implant information (e.g. the safety limit values thereof), etc., and safe scanning requirements can also be met by making numerical adjustments on the basis of the current safety limit values.

Further, when there are a plurality of safety limit values for the same scanning parameter, a new limit value is determined by using the most stringent limit value, thereby ensuring safety during the scanning process.

While the present invention has been described in detail with reference to specific embodiments, it would be understood by those skilled in the art that many modifications and variations can be made to the present invention. Therefore, it should be understood that the claims are intended to cover all such modifications and variations within the true spirit and scope of the present invention.

1. A scanning method based on a magnetic resonance system, comprising:
for at least one scanning parameter, acquiring a numerical distribution map determined on the basis of the magnetic resonance system;
determining an implant-related sensitive region of a subject to be scanned;
determining, from the numerical distribution map, a first numerical value corresponding to the sensitive region;
acquiring a limit value of the at least one scanning parameter limited by an implant of the subject;
adjusting the limit value of the at least one scanning parameter on the basis of the first numerical value; and
scanning the subject by using the adjusted limit value of the at least one scanning parameter.

2. The method according to claim 1, further comprising:
acquiring a body model of the subject; and
on the basis of positioning information of the subject in the magnetic resonance system, determining position information of the body model relative to the magnetic resonance system;
wherein determining an implant-related sensitive region of a subject to be scanned comprises: determining the sensitive region from the body model.

3. The method according to claim 2, wherein determining an implant-related sensitive region of a subject to be scanned comprises:
determining, from a plurality of predefined human body regions, a human body region having the most stringent limit value for the at least one scanning parameter; and
determining, from the body model, a region corresponding to the human body region having the most stringent limit value as the sensitive region.

4. The method according to claim 3, wherein the sensitive region comprises: a region where an implant of the subject is located.

5. The method according to claim 4, wherein the sensitive region comprises:
a region where an implant having the most stringent limit value for the at least one scanning parameter among a plurality of implants of the subject is located.

6. The method according to claim 4, wherein determining the sensitive region from the body model comprises:
adding an implant identifier to the body model on the basis of implant information of the subject; and
determining, on the basis of the added implant identifier, the region where an implant of the subject is located.

7. The method according to claim 6, wherein determining, on the basis of the added implant identifier, the region where an implant of the subject is located comprises:
determining, on the basis of edge position information of the implant identifier, the region where an implant is located.

8. The method according to claim 1, further comprising:
determining, from the numerical distribution map, a second numerical value corresponding to a scan center;
wherein adjusting the limit value of the at least one scanning parameter on the basis of the first numerical value comprises:
adjusting the limit value of the at least one scanning parameter on the basis of a relationship between the first numerical value and the second numerical value.

9. The method according to claim 7, wherein the relationship is a proportional relationship between the first numerical value and the second numerical value.

10. The method according to claim 8, wherein the adjusted limit value of the at least one scanning parameter is the product of the ratio between the first numerical value and the second numerical value, and the limit value before the adjustment.

11. The method according to claim 2, further comprising:
on the basis of predetermined positioning information of the subject in the magnetic resonance system, acquiring a position correspondence between the body model and the numerical distribution map; wherein the first numerical value is determined on the basis of the position correspondence.

12. The method according to claim 1, wherein the at least one scanning parameter comprises a radio-frequency field strength or a SAR, and the numerical distribution map comprises a radio-frequency field map or a SAR distribution map of the magnetic resonance system.

13. The method according to claim 1, wherein
the first numerical value comprises: a maximum numerical value among numerical values corresponding to the sensitive region in the numerical distribution map.

14. The method according to claim 1, wherein the at least one scanning parameter comprises a magnetic field change rate or a maximum gradient slew rate, and the numerical distribution map comprises a magnetic field change rate distribution map of the magnetic resonance system.

15. The method according to claim 14, further comprising:
acquiring reference information, wherein the reference information comprises externally defined magnetic field change rate distribution information, and the magnetic field change rate distribution information comprises a magnetic field change rate in a defined spatial coordinate system, the defined spatial coordinate system corresponding to a magnetic field spatial coordinate system of the magnetic resonance system; and
determining, from the reference information, a second numerical value of the magnetic field change rate corresponding to the sensitive region;
wherein adjusting the limit value of the at least one scanning parameter on the basis of the first numerical value comprises:
adjusting the limit value of the at least one scanning parameter on the basis of a relationship between the first numerical value and the second numerical value.

16. The method according to claim 15, wherein the numerical distribution map comprises a magnetic field change rate distribution map of one or more gradient axes of the magnetic resonance system, and the reference information comprises defined magnetic field change rate distribution information of the one or more gradient axes.

17. The method according to claim 16, wherein
the first numerical value comprises one or more first-axis numerical values, and each of the first-axis numerical values is: a maximum numerical value determined from a numerical region of a magnetic field change rate distribution map of one gradient axis of the magnetic resonance system, the numerical region corresponding to the sensitive region;
the second numerical value comprises one or more second-axis numerical values respectively determined from the defined magnetic field change rate distribution information of the one or more gradient axes, the second-axis numerical values corresponding to the sensitive region; and
adjusting the limit value of the at least one scanning parameter on the basis of the first numerical value comprises:
determining the ratios of the one or more second-axis numerical values to the corresponding one or more first-axis numerical values, respectively, so as to acquire one or more ratio values; and
adjusting, on the basis of the one or more ratio values, a limit value of the magnetic field change rate or the maximum gradient slew rate of the one or more gradient axes of the magnetic resonance system.

18. The method according to claim 17, wherein adjusting, on the basis of the one or more ratio values, a limit value of the magnetic field change rate or the maximum gradient slew rate of the one or more gradient axes of the magnetic resonance system comprises:
determining a minimum value among the one or more ratio values, and
adjusting, on the basis of the minimum value, the limit value of the magnetic field change rate or the maximum gradient slew rate of the one or more gradient axes of the magnetic resonance system.

19. The method according to claim 17, wherein adjusting, on the basis of the one or more ratio values, a limit value of the magnetic field change rate or the maximum gradient slew rate of the one or more gradient axes of the magnetic resonance system comprises:
adjusting, on the basis of the one or more ratio values, a limit value of the magnetic field change rate or the maximum gradient slew rate of one or more corresponding gradient axes of the magnetic resonance system, respectively.

20. A magnetic resonance system, comprising:
a magnetic resonance assembly; and
a controller, configured to control the magnetic resonance assembly to scan a subject to be scanned, and to perform the scanning method according to claim 1.
